(19) United States
(12) Patent Application Publication
(10) Pub. No.: US 2025/0000808 A1
(43) Pub. Date: Jan. 2, 2025

(54) DESIGNER EXTRACELLULAR VESICLES FOR TARGETED DELIVERY TO SCHWANN CELLS

(71) Applicant: Ohio State Innovation Foundation, Columbus, OH (US)

(72) Inventors: Natalia Higuita-Castro, Columbus, OH (US); Daniel Gallego-Perez, Columbus, OH (US); Ana Salazar-Puerta, Columbus, OH (US); Daniel Dodd, Columbus, OH (US)

(21) Appl. No.: 18/704,758

(22) PCT Filed: Oct. 26, 2022
(86) PCT No.: PCT/US2022/078715

§ 371 Date: Apr. 25, 2024

Related U.S. Application Data
(60) Provisional application No. 63/271,912, filed on Oct. 26, 2021.

(51) Int. Cl. A61K 9/50; A61K 38/17

(52) U.S. Cl. CPC A61K 9/5068; A61K 38/1709

(57) ABSTRACT

Disclosed herein are designer extracellular vesicles (EVs) that target Schwann cells (SCs). For example, in some embodiments, the EVs are decorated with HRG1/2, NRG1, or a combination thereof. These EVs can in some embodiments, be used to deliver diagnostic and/or therapeutic cargo to Schwann cells in a subject in need thereof.

### CROSS-REFERENCE TO RELATED APPLICATIONS

This application claims benefit of U.S. Provisional Application No. 63/271,912, filed Oct. 26, 2021, which is hereby incorporated herein by reference in its entirety.

### SEQUENCE LISTING

This application contains a sequence listing filed in ST.26 format entitled “321501_2590_sequence_listing” created on Oct. 25, 2022. The content of the sequence listing is incorporated herein in its entirety.

### BACKGROUND

Neurofibromatosis type I (NF1) is an autosomal dominant genetic condition caused by mutations in the neurofibromin 1 (NF1) gene in Schwann cells (SCs). NF1 is characterized by peripheral nervous system tumors (PNSTs), including plexiform neurofibromas (pNFs) that cause nerve dysfunction, deformity, pain damage to adjacent structures, and can undergo malignant transformation. There are currently no effective therapies to prevent or treat pNFs. Moreover, there is a need for cell-specific delivery to SCs for diagnostic and therapeutic purposes.

### SUMMARY

Disclosed herein are designer extracellular vesicles (EVs) that target Schwann cells (SCs). For example, in some embodiments, the EVs are decorated with NRG1, NRG2, or a combination thereof. Other embodiments may incorporate Schwann cell molecules such as purinergic receptors (I.e., P2X4R) or RTKs such as ErbB3. These EVs can, in some embodiments, be used to deliver diagnostic and/or therapeutic cargo to Schwann cells in a subject in need thereof.

Therefore, also disclosed herein is a method for treating any disease or condition associated with Schwann cells. Schwann cell associated diseases, and potential therapeutic molecules for treatment include Neurofibromatosis type 1 (NF1) and the neurofibromin-1 gene; Neurofibromatosis type 2 (NF2) and the neurofibromin-2 gene; Charcot-Marie-Tooth disease (CMT) and targets that inhibit PMP22 expression; Guillain-Barré syndrome (GBS, acute inflammatory demyelinating polyradiculopathy type), and schwannomatosis by targeting the SMARCB1 or LZTR1 tumor suppressor genes. Chronic inflammatory demyelinating polyneuropathy (CIDP), leprosy, and Zika Virus are all neuropathies involving Schwann cells. Therefore, the disclosed EVs can be used to treat one or more of these neuropathies

In some embodiments, these EVs are loaded with functional neurofibromin 1 and can therefore be used to treat NF1 in a subject. EVs may also be loaded with siRNA or RNAi molecules that target Ras pathway genes to inhibit dysregulated cellular proliferation. Small molecule inhibitors, such as Dabrafenib, Selumetinib, or Nilotinib could also be used in this case. Therefore, also disclosed herein are methods of treating NF1 in a subject that involves engineering the cells of the subject to produce therapeutic EVs that target and deliver functional neurofibromin 1 to Schwann cells (SCs). Also disclosed are methods of collecting EVs produced ex vivo and loading them with neurofibromin 1 for use in treating NF1 in a subject.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates designer EVs with enhanced tropism for SCs will be made ex vivo and deployed in vivo to treat neurofibromas. TNT will be used to turn the epidermis into a designer EV bioreactor to target SCs and treat neurofibromas.

FIGS. 2A and 2B illustrate a tunneling nanotubes (TNT) platform (1: plasmid reservoir, 2: negative and 3: positive lead). FIG. 2C is an electron micrograph. FIGS. 2D to 2F show pulsed fields porate and electrophoretically drive cargo into skin. FIGS. 2G and 2H are simulations showing focused (solid) vs. widespread (dashed) poration in TNT vs. BEP. FIG. 2I shows gene expression vs. BEP. * p<0.05.

FIGS. 3A and 3B show generation of EVs decorated with the SC-targeting ligands NRG1 and NRG2.

FIG. 4 shows percentage uptake of EVs decorated with the SC-targeting ligands NRG1 and NRG2 by PMEFs and SCs.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins-Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, the term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

A “nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software.

By “specifically hybridizes” is meant that a probe, primer, or oligonucleotide recognizes and physically interacts (that is, base-pairs) with a substantially complementary nucleic acid (for example, a c-met nucleic acid) under high stringency conditions, and does not substantially base pair with other nucleic acids.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

The “control elements” or “regulatory sequences” are those non-translated regions of the vector-enhancers, promoters, 5′ and 3′ untranslated regions-which interact with host cellular proteins to carry out transcription and translation. Such elements may vary in their strength and specificity.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

### Schwann Cell Targeting Extracellular Vehicles (EVs)

Disclosed herein are EVs that target Schwann cells (SCs) that can be loaded with therapeutic and/or diagnostic cargo. In some embodiments, the method involves engineering cells of the subject to produce therapeutic EVs. In some embodiments, the method involves collecting EVs produced ex vivo and loading them with therapeutic cargo.

### Engineering Patient Cells to Produce Therapeutic EVs

Disclosed are methods of reprogramming cells of the subject into EV-producing cells that involve delivering intracellularly into cells a polynucleotide comprising nucleic acid sequences encoding SC targeting ligands and optionally a therapeutic cargo. In some embodiments, the cells can be any cell in the subject able to produce EVs, including (but not limited to) skin cells (e.g., fibroblasts, keratinocytes, skin stem cells), adipocytes, dendritic cells, peripheral blood mononuclear cells (PBMC), pancreatic cells (e.g., ductal epithelial cells), liver cells (e.g., hepatocytes), immune cells (e.g., T cells, macrophages, myeloid derived suppressor cells).

For example, disclosed herein are compositions and methods for reprogramming skin cells into EV-producing cells both in vitro and in vivo that can be used to treat NF1.

In some embodiments, this method involves transfecting the cells of the subject with an expression vector encoding NRG1, NRG2, or any combination thereof. In some embodiments, this method involves transfecting the cells of the subject with an expression vector encoding neurofibromin.

The mRNA sequence for mouse NRG1 is provided in NCBI Accession No. NM_178591.3, which is incorporated by reference for this sequence. The mRNA sequence for mouse NRG2 is provided in NCBI Accession No. NM_001167891.3, which is incorporated by reference for this sequence.

In some embodiments, a human NRG1 cDNA has the nucleic acid sequence:

(SEQ ID NO: 1, BC150609.1)
GAGCCCTTGGACCAAACTCGCCTGCGCCGAGAGCCGTCCGCGTAG
AGCGCTCCGTCTCCGGCGAGATGTCCGAGCGCAAAGAAGGCAGAG
GCAAAGGGAAGGGCAAGAAGAAGGAGCGAGGCTCCGGCAAGAAGC
CGGAGTCCGCGGCGGGCAGCCAGAGCCCAGCCTTGCCTCCCCAAT
TGAAAGAGATGAAAAGCCAGGAATCGGCTGCAGGTTCCAAACTAG
TCCTTCGGTGTGAAACCAGTTCTGAATACTCCTCTCTCAGATTCA
AGTGGTTCAAGAATGGGAATGAATTGAATCGAAAAAACAAACCAC
AAAATATCAAGATACAAAAAAAGCCAGGGAAGTCAGAACTTCGCA
TTAACAAAGCATCACTGGCTGATTCTGGAGAGTATATGTGCAAAG
TGATCAGCAAATTAGGAAATGACAGTGCCTCTGCCAATATCACCA
TCGTGGAATCAAACGAGATCATCACTGGTATGCCAGCCTCAACTG
AAGGAGCATATGTGTCTTCAGAGTCTCCCATTAGAATATCAGTAT
CCACAGAAGGAGCAAATACTTCTTCATCTACATCTACATCCACCA
CTGGGACAAGCCATCTTGTAAAATGTGCGGAGAAGGAGAAAACTT
TCTGTGTGAATGGAGGGGAGTGCTTCATGGTGAAAGACCTTTCAA
ACCCCTCGAGATACTTGTGCAAGTGCCAACCTGGATTCACTGGAG
CAAGATGTACTGAGAATGTGCCCATGAAAGTCCAAAACCAAGAAA
AGGCGGAGGAGCTGTACCAGAAGAGAGTGCTGACCATAACCGGCA
TCTGCATCGCCCTCCTTGTGGTCGGCATCATGTGTTTGGTGGCCT
ACTGCAAAACCAAGAAACAGCGGAAAAAGCTGCATGACCGTCTTC
GGCAGAGCCTTCGGTCTGAACGAAACAATATGATGAACATTGCCA
ATGGGCCTCACCATCCTAACCCACCCCCCGAGAATGTCCAGCTGG
TGAATCAATACGTATCTAAAAACGTCATCTCCAGTGAGCATATTG
TTGAGAGAGAAGCAGAGACATCCTTTTCCACCAGTCACTATACTT
CCACAGCCCATCACTCCACTACTGTCACCCAGACTCCTAGCCACA
GCTGGAGCAACGGACACACTGAAAGCATCCTTTCCGAAAGCCACT
CTGTAATCGTGATGTCATCCGTAGAAAACAGTAGGCACAGCAGCC
CAACTGGGGGCCCAAGAGGACGTCTTAATGGCACAGGAGGCCCTC
GTGAATGTAACAGCTTCCTCAGGCATGCCAGAGAAACCCCTGATT
CCTACCGAGACTCTCCTCATAGTGAAAGGTATGTGTCAGCCATGA
CCACCCCGGCTCGTATGTCACCTGTAGATTTCCACACGCCAAGCT
CCCCCAAATCGCCCCCTTCGGAAATGTCTCCACCCGTGTCCAGCA
TGACGGTGTCCATGCCTTCCATGGCGGTCAGCCCCTTCATGGAAG
AAGAGAGACCTCTACTTCTCGTGACACCACCAAGGCTGCGGGAGA
AGAAGTTTGACCATCACCCTCAGCAGTTCAGCTCCTTCCACCACA
ACCCCGCGCATGACAGTAACAGCCTCCCTGCTAGCCCCTTGAGGA
TAGTGGAGGATGAGGAGTATGAAACGACCCAAGAGTACGAGCCAG
CCCAAGAGCCTGTTAAGAAACTCGCCAATAGCCGGCGGGCCAAAA
GAACCAAGCCCAATGGCCACATTGCTAACAGATTGGAAGTGGACA
GCAACACAAGCTCCCAGAGCAGTAACTCAGAGAGTGAAACAGAAG
ATGAAAGAGTAGGTGAAGATACGCCTTTCCTGGGCATACAGAACC
CCCTGGCAGCCAGTCTTGAGGCAACACCTGCCTTCCGCCTGGCTG
ACAGCAGGACTAACCCAGCAGGCCGCTTCTCGACACAGGAAGAAA
TCCAGGCCAGGCTGTCTAGTGTAATTGCTAACCAAGACCCTATTG
CTGTATAAAACCTAAATAAACACATAGATTCACCTGTAAAACTTT
ATTTTATATAATAAAGTATTCCACCTTAAATTAAACAATTTATTT
TATTTTAGCAGTTCTGCAAATAGAAAACAGGAAAAA.

In some embodiments, the human NRG1 mRNA encodes the amino acid sequence:

(SEQ ID NO: 2, AAI50610.1)
MSERKEGRGKGKGKKKERGSGKKPESAAGSQSPALPPQLKEMKSQ
ESAAGSKLVLRCETSSEYSSLRFKWFKNGNELNRKNKPQNIKIQK
KPGKSELRINKASLADSGEYMCKVISKLGNDSASANITIVESNEI
ITGMPASTEGAYVSSESPIRISVSTEGANTSSSTSTSTTGTSHLV
KCAEKEKTFCVNGGECFMVKDLSNPSRYLCKCQPGFTGARCTENV
PMKVQNQEKAEELYQKRVLTITGICIALLVVGIMCLVAYCKTKKQ
RKKLHDRLRQSLRSERNNMMNIANGPHHPNPPPENVQLVNQYVSK
NVISSEHIVEREAETSFSTSHYTSTAHHSTTVTQTPSHSWSNGHT
ESILSESHSVIVMSSVENSRHSSPTGGPRGRLNGTGGPRECNSFL
RHARETPDSYRDSPHSERYVSAMTTPARMSPVDFHTPSSPKSPPS
EMSPPVSSMTVSMPSMAVSPFMEEERPLLLVTPPRLREKKFDHHP
QQFSSFHHNPAHDSNSLPASPLRIVEDEEYETTQEYEPAQEPVKK
LANSRRAKRTKPNGHIANRLEVDSNTSSQSSNSESETEDERVGED
TPFLGIQNPLAASLEATPAFRLADSRTNPAGRFSTQEEIQARLSS
VIANQDPIAV.

In some embodiments, the human NRG2 cDNA has the nucleic acid sequence:

(SEQ ID NO: 3, BC166615.1)
GTACAAAAAAGCAGAAGGGCCGTCAAGGCCCACCATGCGGCAGGT
TTGCTGCTCAGCGCTGCCGCCGCCGCCACTGGAGAAGGGTCGGTG
CAGCAGCTACAGCGACAGCAGCAGCAGCAGCAGCGAGAGGAGCAG
CAGCAGCAGCAGCAGCAGCAGCGAGAGCGGCAGCAGCAGCAGGAG
CAGCAGCAACAACAGCAGCATCTCTCGTCCCGCTGCGCCCCCAGA
GCCGCGGCCGCAGCAACAGCCGCAGCCCCGCAGCCCCGCAGCCCG
GAGAGCCGCCGCCCGTTCGCGAGCCGCAGCCGCCGGCGGCATGAG
GCGCGACCCGGCCCCCGGCTTCTCCATGCTGCTCTTCGGTGTGTC
GCTCGCCTGCTACTCGCCCAGCCTCAAGTCAGTGCAGGACCAGGC
GTACAAGGCACCCGTGGTGGTGGAGGGCAAGGTACAGGGGCTGGT
CCCAGCCGGCGGCTCCAGCTCCAACAGCACCCGAGAGCCGCCCGC
CTCGGGTCGGGTGGCGTTGGTAAAGGTGCTGGACAAGTGGCCGCT
CCGGAGCGGGGGGCTGCAGCGCGAGCAGGTGATCAGCGTGGGCTC
CTGTGTGCCGCTCGAAAGGAACCAGCGCTACATCTTTTTCCTGGA
GCCCACGGAACAGCCCTTAGTCTTTAAGACGGCCTTTGCCCCCCT
CGATACCAACGGCAAAAATCTCAAGAAAGAGGTGGGCAAGATCCT
GTGCACTGACTGCGCCACCCGGCCCAAGTTGAAGAAGATGAAGAG
CCAGACGGGACAGGTGGGTGAGAAGCAATCGCTGAAGTGTGAGGC
AGCAGCCGGTAATCCCCAGCCTTCCTACCGTTGGTTCAAGGATGG
CAAGGAGCTCAACCGCAGCCGAGACATTCGCATCAAATATGGCAA
CGGCAGAAAGAACTCACGACTACAGTTCAACAAGGTGAAGGTGGA
GGACGCTGGGGAGTATGTCTGCGAGGCCGAGAACATCCTGGGGAA
GGACACCGTCCGGGGCCGGCTTTACGTCAACAGCGTGAGCACCAC
CCTGTCATCCTGGTCGGGGCACGCCCGGAAGTGCAACGAGACAGC
CAAGTCCTATTGCGTCAATGGAGGCGTCTGCTACTACATCGAGGG
CATCAACCAGCTCTCCTGCAAGTGTCCTGTGGGATACACCGGGGA
CAGGTGTCAGCAGTTCGCAATGGTCAACTTCTCCAAGCACCTTGG
ATTTGAATTAAAGGAAGCCGAGGAGCTGTACCAGAAGAGGGTCCT
GACCATCACGGGCATCTGCGTGGCTCTGCTGGTCGTGGGCATCGT
CTGTGTGGTGGCCTACTGCAAGACCAAAAAACAGCGGAAGCAGAT
GCACAACCACCTCCGGCAGAACATGTGCCCGGCCCATCAGAACCG
GAGCTTGGCCAATGGGCCCAGCCACCCCCGGCTGGACCCAGAGGA
GATCCAGATGGCAGATTATATTTCCAAGAACGTGCCAGCCACAGA
CCATGTCATCAGGAGAGAAACTGAGACCACCTTCTCTGGGAGCCA
CTCCTGTTCTCCTTCTCACCACTGCTCCACAGCCACACCCACCTC
CAGCCACAGACACGAGAGCCACACGTGGAGCCTGGAACGTTCTGA
GAGCCTGACTTCTGACTCCCAGTCGGGGATCATGCTATCATCAGT
GGGTACCAGCAAATGCAACAGCCCAGCATGTGTGGAGGCCCGGGC
AAGGCGGGCAGCAGCCTACAACCTGGAGGAGCGGCGCAGGGCCAC
CGCGCCACCCTATCACGATTCCGTGGACTCCCTTCGCGACTCCCC
ACACAGCGAGAGGTACGTGTCGGCCCTGACCACGCCCGCGCGCCT
CTCGCCCGTGGACTTCCACTACTCGCTGGCCACGCAGGTGCCAAC
TTTCGAGATCACGTCCCCCAACTCGGCGCACGCCGTGTCGCTGCC
GCCGGCGGCGCCCATCAGTTACCGCCTGGCCGAGCAGCAGCCGTT
ACTGCGGCACCCGGCGCCCCCCGGCCCGGGACCCGGACCCGGGCC
CGGGCCCGGGCCCGGCGCAGACATGCAGCGCAGCTATGACAGCTA
CTATTACCCCGCGGCGGGGCCCGGACCGCGGCGCGGGACCTGCGC
GCTCGGCGGCAGCCTGGGCAGCCTGCCTGCCAGCCCCTTCCGCAT
CCCCGAGGACGACGAGTACGAGACCACGCAGGAGTGCGCGCCCCC
GCCGCCGCCGCGGCCGCGCGCGCGCGGTGCGTCCCGCAGGACGTC
GGCGGGGCCCCGGCGCTGGCGCCGCTCGCGCCTCAACGGGCTGGC
GGCGCAGCGCGCACGGGCGGCGAGGGACTCGCTGTCGCTGAGCAG
CGGCTCGGGCGGCGGCTCAGCCTCGGCGTCGGACGACGACGCGGA
CGACGCGGACGGGGCGCTGGCGGCCGAGAGCACACCTTTCCTGGG
CCTGCGTGGGGCGCACGACGCGCTGCGCTCGGACTCGCCGCCACT
GTGCCCGGCGGCCGACAGCAGGACTTACTACTCACTGGACAGCCA
CAGCACGCGGGCCAGCAGCAGACACAGCCGCGGGCCGCCCCCGCG
GGCCAAGCAGGACTCGGCGCCACTCTAGGGCCTCATGGGCCCAGC
TTTCTTGTAC.

In some embodiments, the human NRG2 mRNA encodes the amino acid sequence:

(SEQ ID NO: 4, AAI66615.1)
MRQVCCSALPPPPLEKGRCSSYSDSSSSSSERSSSSSSSSSESGS
SSRSSSNNSSISRPAAPPEPRPQQQPQPRSPAARRAAARSRAAAA
GGMRRDPAPGFSMLLFGVSLACYSPSLKSVQDQAYKAPVVVEGKV
QGLVPAGGSSSNSTREPPASGRVALVKVLDKWPLRSGGLQREQVI
SVGSCVPLERNQRYIFFLEPTEQPLVFKTAFAPLDTNGKNLKKEV
GKILCTDCATRPKLKKMKSQTGQVGEKQSLKCEAAAGNPQPSYRW
FKDGKELNRSRDIRIKYGNGRKNSRLQFNKVKVEDAGEYVCEAEN
ILGKDTVRGRLYVNSVSTTLSSWSGHARKCNETAKSYCVNGGVCY
YIEGINQLSCKCPVGYTGDRCQQFAMVNFSKHLGFELKEAEELYQ
KRVLTITGICVALLVVGIVCVVAYCKTKKQRKQMHNHLRQNMCPA
HQNRSLANGPSHPRLDPEEIQMADYISKNVPATDHVIRRETETTF
SGSHSCSPSHHCSTATPTSSHRHESHTWSLERSESLTSDSQSGIM
LSSVGTSKCNSPACVEARARRAAAYNLEERRRATAPPYHDSVDSL
RDSPHSERYVSALTTPARLSPVDFHYSLATQVPTFEITSPNSAHA
VSLPPAAPISYRLAEQQPLLRHPAPPGPGPGPGPGPGPGADMQRS
YDSYYYPAAGPGPRRGTCALGGSLGSLPASPFRIPEDDEYETTQE
CAPPPPPRPRARGASRRTSAGPRRWRRSRLNGLAAQRARAARDSL
SLSSGSGGGSASASDDDADDADGALAAESTPFLGLRGAHDALRSD
SPPLCPAADSRTYYSLDSHSTRASSRHSRGPPPRAKQDSAPL.

In some embodiments, the EVs incorporate Schwann cell molecules such as purinergic receptors (I.e., P2X4R) or RTKs such as ErbB3.

In some embodiments, the human purinergic receptor P2X 4 (P2RX4) cDNA has the nucleic acid sequence:

(SEQ ID NO: 5, P2RX4 transcript variant X2)
AGACCGACTAGGGGACTGGGAGCGGGCGGCGCGGCCATGGCGGGC
TGCTGCGCCGCGCTGGCGGCCTTCCTGTTCGAGTACGACACGCCG
CGCATCGTGCTCATCCGCAGCCGCAAAGTGGGGCTCATGAACCGC
GCCGTGCAACTGCTCATCCTGGCCTACGTCATCGGACCTGCTTTT
TTAAAGGCTGCAGAAAACTTCACTCTTTTGGTTAAGAACAACATC
TGGTATCCCAAATTTAATTTCAGCAAGAGGAATATCCTTCCCAAC
ATCACCACTACTTACCTCAAGTCGTGCATTTATGATGCTAAAACA
GATCCCTTCTGCCCCATATTCCGTCTTGGCAAAATAGTGGAGAAC
GCAGGACACAGTTTCCAGGACATGGCCGTGGAGGGAGGCATCATG
GGCATCCAGGTCAACTGGGACTGCAACCTGGACAGAGCCGCCTCC
CTCTGCTTGCCCAGGTACTCCTTCCGCCGCCTCGATACACGGGAC
GTTGAGCACAACGTATCTCCTGGCTACAATTTCAGGTTTGCCAAG
TACTACAGAGACCTGGCTGGCAACGAGCAGCGCACGCTCATCAAG
GCCTATGGCATCCGCTTCGACATCATTGTGTTTGGGAAGGCAGGG
AAATTTGACATCATCCCCACTATGATCAACATCGGCTCTGGCCTG
GCACTGCTAGGCATGGCGACCGTGCTGTGTGACATCATAGTCCTC
TACTGCATGAAGAAAAGACTCTACTATCGGGAGAAGAAATATAAA
TATGTGGAAGATTACGAGCAGGGTCTTGCTAGTGAGCTGGACCAG
TGAGGCCTACCCCACACCTGGGCTCTCCACAGCCCCATCAAAGAA
CAGAGAGGAGGAGGAGGGAGAAATGGCCACCACATCACCCCAGAG
AAATTTCTGGAATCTGATTGAGTCTCCACTCCACAAGCACTCAGG
GTTCCCCAGCAGCTCCTGTGTGTTGTGTGCAGGATCTGTTTGCCC
ACTCGGCCCAGGAGGTCAGCAGTCTGTTCTTGGCTGGGTCAACTC
TGCTTTTCCCGCAACCTGGGGTTGTCGGGGGAGCGCTGGCCCGAC
GCAGTGGCACTGCTGTGGCTTTCAGGGCTGGAGCTGGCTTTGCTC
AGAAGCCTCCTGTCTCCAGCTCTCTCCAGGACAGGCCCAGTCCTC
TGAGGCACGGCGGCTCTGTTCAAGCACTTTATGCGGCAGGGGAGG
CCGCCTGGCTGCAGTCACTAGACTTGTAGCAGGCCTGGGCTGCAG
GCTTCCCCCCGACCATTCCCTGCAGCCATGCGGCAGAGCTGGCAT
TTCTCCTCAGAGAAGCGCTGTGCTAAGGTGATCGAGGACCAGACA
TTAAAGCGTGATTTTCTTAA.

In some embodiments, the human P2RX4 mRNA encodes the amino acid sequence:

(SEQ ID NO: 6)
MAGCCAALAAFLFEYDTPRIVLIRSRKVGLMNRAVQLLILAYVIG
PAFLKAAENFTLLVKNNIWYPKFNFSKRNILPNITTTYLKSCIYD
AKTDPFCPIFRLGKIVENAGHSFQDMAVEGGIMGIQVNWDCNLDR
AASLCLPRYSFRRLDTRDVEHNVSPGYNFRFAKYYRDLAGNEQRT
LIKAYGIRFDIIVFGKAGKFDIIPTMINIGSGLALLGMATVLCDI
IVLYCMKKRLYYREKKYKYVEDYEQGLASELDQ.

In some embodiments, the human erb-b2 receptor tyrosine kinase 3 (ERBB3) cDNA has the nucleic acid sequence:

(SEQ ID NO: 7, ERBB3 transcript variant 1)
GCAATTTGCAACCTCCGCTGCCGTCGCCGCAGCAGCCACCAATTC
GCCAGCGGTTCAGGTGGCTCTTGCCTCGATGTCCTAGCCTAGGGG
CCCCCGGGCCGGACTTGGCTGGGCTCCCTTCACCCTCTGCGGAGT
CATGAGGGCGAACGACGCTCTGCAGGTGCTGGGCTTGCTTTTCAG
CCTGGCCCGGGGCTCCGAGGTGGGCAACTCTCAGGCAGTGTGTCC
TGGGACTCTGAATGGCCTGAGTGTGACCGGCGATGCTGAGAACCA
ATACCAGACACTGTACAAGCTCTACGAGAGGTGTGAGGTGGTGAT
GGGGAACCTTGAGATTGTGCTCACGGGACACAATGCCGACCTCTC
CTTCCTGCAGTGGATTCGAGAAGTGACAGGCTATGTCCTCGTGGC
CATGAATGAATTCTCTACTCTACCATTGCCCAACCTCCGCGTGGT
GCGAGGGACCCAGGTCTACGATGGGAAGTTTGCCATCTTCGTCAT
GTTGAACTATAACACCAACTCCAGCCACGCTCTGCGCCAGCTCCG
CTTGACTCAGCTCACCGAGATTCTGTCAGGGGGTGTTTATATTGA
GAAGAACGATAAGCTTTGTCACATGGACACAATTGACTGGAGGGA
CATCGTGAGGGACCGAGATGCTGAGATAGTGGTGAAGGACAATGG
CAGAAGCTGTCCCCCCTGTCATGAGGTTTGCAAGGGGCGATGCTG
GGGTCCTGGATCAGAAGACTGCCAGACATTGACCAAGACCATCTG
TGCTCCTCAGTGTAATGGTCACTGCTTTGGGCCCAACCCCAACCA
GTGCTGCCATGATGAGTGTGCCGGGGGCTGCTCAGGCCCTCAGGA
CACAGACTGCTTTGCCTGCCGGCACTTCAATGACAGTGGAGCCTG
TGTACCTCGCTGTCCACAGCCTCTTGTCTACAACAAGCTAACTTT
CCAGCTGGAACCCAATCCCCACACCAAGTATCAGTATGGAGGAGT
TTGTGTAGCCAGCTGTCCCCATAACTTTGTGGTGGATCAAACATC
CTGTGTCAGGGCCTGTCCTCCTGACAAGATGGAAGTAGATAAAAA
TGGGCTCAAGATGTGTGAGCCTTGTGGGGGACTATGTCCCAAAGC
CTGTGAGGGAACAGGCTCTGGGAGCCGCTTCCAGACTGTGGACTC
GAGCAACATTGATGGATTTGTGAACTGCACCAAGATCCTGGGCAA
CCTGGACTTTCTGATCACCGGCCTCAATGGAGACCCCTGGCACAA
GATCCCTGCCCTGGACCCAGAGAAGCTCAATGTCTTCCGGACAGT
ACGGGAGATCACAGGTTACCTGAACATCCAGTCCTGGCCGCCCCA
CATGCACAACTTCAGTGTTTTTTCCAATTTGACAACCATTGGAGG
CAGAAGCCTCTACAACCGGGGCTTCTCATTGTTGATCATGAAGAA
CTTGAATGTCACATCTCTGGGCTTCCGATCCCTGAAGGAAATTAG
TGCTGGGCGTATCTATATAAGTGCCAATAGGCAGCTCTGCTACCA
CCACTCTTTGAACTGGACCAAGGTGCTTCGGGGGCCTACGGAAGA
GCGACTAGACATCAAGCATAATCGGCCGCGCAGAGACTGCGTGGC
AGAGGGCAAAGTGTGTGACCCACTGTGCTCCTCTGGGGGATGCTG
GGGCCCAGGCCCTGGTCAGTGCTTGTCCTGTCGAAATTATAGCCG
AGGAGGTGTCTGTGTGACCCACTGCAACTTTCTGAATGGGGAGCC
TCGAGAATTTGCCCATGAGGCCGAATGCTTCTCCTGCCACCCGGA
ATGCCAACCCATGGAGGGCACTGCCACATGCAATGGCTCGGGCTC
TGATACTTGTGCTCAATGTGCCCATTTTCGAGATGGGCCCCACTG
TGTGAGCAGCTGCCCCCATGGAGTCCTAGGTGCCAAGGGCCCAAT
CTACAAGTACCCAGATGTTCAGAATGAATGTCGGCCCTGCCATGA
GAACTGCACCCAGGGGTGTAAAGGACCAGAGCTTCAAGACTGTTT
AGGACAAACACTGGTGCTGATCGGCAAAACCCATCTGACAATGGC
TTTGACAGTGATAGCAGGATTGGTAGTGATTTTCATGATGCTGGG
CGGCACTTTTCTCTACTGGCGTGGGCGCCGGATTCAGAATAAAAG
GGCTATGAGGCGATACTTGGAACGGGGTGAGAGCATAGAGCCTCT
GGACCCCAGTGAGAAGGCTAACAAAGTCTTGGCCAGAATCTTCAA
AGAGACAGAGCTAAGGAAGCTTAAAGTGCTTGGCTCGGGTGTCTT
TGGAACTGTGCACAAAGGAGTGTGGATCCCTGAGGGTGAATCAAT
CAAGATTCCAGTCTGCATTAAAGTCATTGAGGACAAGAGTGGACG
GCAGAGTTTTCAAGCTGTGACAGATCATATGCTGGCCATTGGCAG
CCTGGACCATGCCCACATTGTAAGGCTGCTGGGACTATGCCCAGG
GTCATCTCTGCAGCTTGTCACTCAATATTTGCCTCTGGGTTCTCT
GCTGGATCATGTGAGACAACACCGGGGGGCACTGGGGCCACAGCT
GCTGCTCAACTGGGGAGTACAAATTGCCAAGGGAATGTACTACCT
TGAGGAACATGGTATGGTGCATAGAAACCTGGCTGCCCGAAACGT
GCTACTCAAGTCACCCAGTCAGGTTCAGGTGGCAGATTTTGGTGT
GGCTGACCTGCTGCCTCCTGATGATAAGCAGCTGCTATACAGTGA
GGCCAAGACTCCAATTAAGTGGATGGCCCTTGAGAGTATCCACTT
TGGGAAATACACACACCAGAGTGATGTCTGGAGCTATGGTGTGAC
AGTTTGGGAGTTGATGACCTTCGGGGCAGAGCCCTATGCAGGGCT
ACGATTGGCTGAAGTACCAGACCTGCTAGAGAAGGGGGAGCGGTT
GGCACAGCCCCAGATCTGCACAATTGATGTCTACATGGTGATGGT
CAAGTGTTGGATGATTGATGAGAACATTCGCCCAACCTTTAAAGA
ACTAGCCAATGAGTTCACCAGGATGGCCCGAGACCCACCACGGTA
TCTGGTCATAAAGAGAGAGAGTGGGCCTGGAATAGCCCCTGGGCC
AGAGCCCCATGGTCTGACAAACAAGAAGCTAGAGGAAGTAGAGCT
GGAGCCAGAACTAGACCTAGACCTAGACTTGGAAGCAGAGGAGGA
CAACCTGGCAACCACCACACTGGGCTCCGCCCTCAGCCTACCAGT
TGGAACACTTAATCGGCCACGTGGGAGCCAGAGCCTTTTAAGTCC
ATCATCTGGATACATGCCCATGAACCAGGGTAATCTTGGGGAGTC
TTGCCAGGAGTCTGCAGTTTCTGGGAGCAGTGAACGGTGCCCCCG
TCCAGTCTCTCTACACCCAATGCCACGGGGATGCCTGGCATCAGA
GTCATCAGAGGGGCATGTAACAGGCTCTGAGGCTGAGCTCCAGGA
GAAAGTGTCAATGTGTAGGAGCCGGAGCAGGAGCCGGAGCCCACG
GCCACGCGGAGATAGCGCCTACCATTCCCAGCGCCACAGTCTGCT
GACTCCTGTTACCCCACTCTCCCCACCCGGGTTAGAGGAAGAGGA
TGTCAACGGTTATGTCATGCCAGATACACACCTCAAAGGTACTCC
CTCCTCCCGGGAAGGCACCCTTTCTTCAGTGGGTCTCAGTTCTGT
CCTGGGTACTGAAGAAGAAGATGAAGATGAGGAGTATGAATACAT
GAACCGGAGGAGAAGGCACAGTCCACCTCATCCCCCTAGGCCAAG
TTCCCTTGAGGAGCTGGGTTATGAGTACATGGATGTGGGGTCAGA
CCTCAGTGCCTCTCTGGGCAGCACACAGAGTTGCCCACTCCACCC
TGTACCCATCATGCCCACTGCAGGCACAACTCCAGATGAAGACTA
TGAATATATGAATCGGCAACGAGATGGAGGTGGTCCTGGGGGTGA
TTATGCAGCCATGGGGGCCTGCCCAGCATCTGAGCAAGGGTATGA
AGAGATGAGAGCTTTTCAGGGGCCTGGACATCAGGCCCCCCATGT
CCATTATGCCCGCCTAAAAACTCTACGTAGCTTAGAGGCTACAGA
CTCTGCCTTTGATAACCCTGATTACTGGCATAGCAGGCTTTTCCC
CAAGGCTAATGCCCAGAGAACGTAACTCCTGCTCCCTGTGGCACT
CAGGGAGCATTTAATGGCAGCTAGTGCCTTTAGAGGGTACCGTCT
TCTCCCTATTCCCTCTCTCTCCCAGGTCCCAGCCCCTTTTCCCCA
GTCCCAGACAATTCCATTCAATCTTTGGAGGCTTTTAAACATTTT
GACACAAAATTCTTATGGTATGTAGCCAGCTGTGCACTTTCTTCT
CTTTCCCAACCCCAGGAAAGGTTTTCCTTATTTTGTGTGCTTTCC
CAGTCCCATTCCTCAGCTTCTTCACAGGCACTCCTGGAGATATGA
AGGATTACTCTCCATATCCCTTCCTCTCAGGCTCTTGACTACTTG
GAACTAGGCTCTTATGTGTGCCTTTGTTTCCCATCAGACTGTCAA
GAAGAGGAAAGGGAGGAAACCTAGCAGAGGAAAGTGTAATTTTGG
TTTATGACTCTTAACCCCCTAGAAAGACAGAAGCTTAAAATCTGT
GAAGAAAGAGGTTAGGAGTAGATATTGATTACTATCATAATTCAG
CACTTAACTATGAGCCAGGCATCATACTAAACTTCACCTACATTA
TCTCACTTAGTCCTTTATCATCCTTAAAACAATTCTGTGACATAC
ATATTATCTCATTTTACACAAAGGGAAGTCGGGCATGGTGGCTCA
TGCCTGTAATCTCAGCACTTTGGGAGGCTGAGGCAGAAGGATTAC
CTGAGGCAAGGAGTTTGAGACCAGCTTAGCCAACATAGTAAGACC
CCCATCTCTTTAAAAAAAAAAAAAAAAAAAAAAAAAAAACTTTAG
AACTGGGTGCAGTGGCTCATGCCTGTAATCCCAGCCAGCACTTTG
GGAGGCTGAGATGGGAAGATCACTTGAGCCCAGAATTAGAGATAA
GCCTATGGAAACATAGCAAGACACTGTCTCTACAGGGGAAAAAAA
AAAAAGAAACTGAGCCTTAAAGAGATGAAATAAATTAAGCAGTAG
ATCCAGGATGCAAAATCCTCCCAATTCCTGTGCATGTGCTCTTAT
TGTAAGGTGCCAAGAAAAACTGATTTAAGTTACAGCCCTTGTTTA
AGGGGCACTGTTTCTTGTTTTTGCACTGAATCAAGTCTAACCCCA
ACAGCCACATCCTCCTATACCTAGACATCTCATCTCAGGAAGTGG
TGGTGGGGGTAGTCAGAAGGAAAAATAACTGGACATCTTTGTGTA
AACCATAATCCACATGTGCCGTAAATGATCTTCACTCCTTATCCG
AGGGCAAATTCACAAGGATCCCCAAGATCCACTTTTAGAAGCCAT
TCTCATCCAGCAGTGAGAAGCTTCCAGGTAGGACAGAAAAAAGAT
CCAGCTTCAGCTGCACACCTCTGTCCCCTTGGATGGGGAACTAAG
GGAAAACGTCTGTTGTATCACTGAAGTTTTTTGTTTTGTTTTTAT
ACGTGTCTGAATAAAAATGCCAAAGTTTTTTTTCA.

In some embodiments, the human ERBB3 mRNA encodes the amino acid sequence:

(SEQ ID NO: 8)
MRANDALQVLGLLFSLARGSEVGNSQAVCPGTLNGLSVTGDAENQ
YQTLYKLYERCEWVMGNLEIVLTGHNADLSFLQWIREVTGYVLVA
MNEFSTLPLPNLRVVRGTQVYDGKFAIFVMLNYNTNSSHALRQLR
LTQLTEILSGGVYIEKNDKLCHMDTIDWRDIVRDRDAEIVVKDNG
RSCPPCHEVCKGRCWGPGSEDCQTLTKTICAPQCNGHCFGPNPNQ
CCHDECAGGCSGPQDTDCFACRHFNDSGACVPRCPQPLVYNKLTF
QLEPNPHTKYQYGGVCVASCPHNFVVDQTSCVRACPPDKMEVDKN
GLKMCEPCGGLCPKACEGTGSGSRFQTVDSSNIDGFVNCTKILGN
LDFLITGLNGDPWHKIPALDPEKLNVFRTVREITGYLNIQSWPPH
MHNFSVFSNLTTIGGRSLYNRGFSLLIMKNLNVTSLGFRSLKEIS
AGRIYISANRQLCYHHSLNWTKVLRGPTEERLDIKHNRPRRDCVA
EGKVCDPLCSSGGCWGPGPGQCLSCRNYSRGGVCVTHCNFLNGEP
REFAHEAECFSCHPECQPMEGTATCNGSGSDTCAQCAHFRDGPHC
VSSCPHGVLGAKGPIYKYPDVQNECRPCHENCTQGCKGPELQDCL
GQTLVLIGKTHLTMALTVIAGLVVIFMMLGGTFLYWRGRRIQNKR
AMRRYLERGESIEPLDPSEKANKVLARIFKETELRKLKVLGSGVF
GTVHKGVWIPEGESIKIPVCIKVIEDKSGRQSFQAVTDHMLAIGS
LDHAHIVRLLGLCPGSSLQLVTQYLPLGSLLDHVRQHRGALGPQL
LLNWGVQIAKGMYYLEEHGMVHRNLAARNVLLKSPSQVQVADFGV
ADLLPPDDKQLLYSEAKTPIKWMALESIHFGKYTHQSDVWSYGVT
VWELMTFGAEPYAGLRLAEVPDLLEKGERLAQPQICTIDVYMVMV
KCWMIDENIRPTFKELANEFTRMARDPPRYLVIKRESGPGIAPGP
EPHGLTNKKLEEVELEPELDLDLDLEAEEDNLATTTLGSALSLPV
GTLNRPRGSQSLLSPSSGYMPMNQGNLGESCQESAVSGSSERCPR
PVSLHPMPRGCLASESSEGHVTGSEAELQEKVSMCRSRSRSRSPR
PRGDSAYHSQRHSLLTPVTPLSPPGLEEEDVNGYVMPDTHLKGTP
SSREGTLSSVGLSSVLGTEEEDEDEEYEYMNRRRRHSPPHPPRPS
SLEELGYEYMDVGSDLSASLGSTQSCPLHPVPIMPTAGTTPDEDY
EYMNRQRDGGGPGGDYAAMGACPASEQGYEEMRAFQGPGHQAPHV
HYARLKTLRSLEATDSAFDNPDYWHSRLFPKANAQRT.

In some embodiments, the nucleic acid sequences are present in non-viral vectors. In some embodiments, the nucleic acid sequences are operably linked to an expression control sequence. In other embodiments the nucleic acids are operably linked to two or more expression control sequences.

A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

In some embodiments, EVs containing the disclosed nucleic acid sequences are administered to the cells of the subject, which can then induce cells in the subject to be EV-producing cells. Therefore, also disclosed is a method of reprogramming cells into EV-producing cells that involves exposing the cell with an extracellular vesicle produced from a cell containing or expressing the disclosed therapeutic genes.

Exosomes and microvesicles are EVs that differ based on their process of biogenesis and biophysical properties, including size and surface protein markers.

Exosomes are homogenous small particles ranging from 40 to 150 nm in size and they are normally derived from the endocytic recycling pathway. In endocytosis, endocytic vesicles form at the plasma membrane and fuse to form early endosomes. These mature and become late endosomes where intraluminal vesicles bud off into an intra-vesicular lumen. Instead of fusing with the lysosome, these multivesicular bodies directly fuse with the plasma membrane and release exosomes into the extracellular space. Exosome biogenesis, protein cargo sorting, and release involve the endosomal sorting complex required for transport (ESCRT complex) and other associated proteins such as Alix and Tsg101. In contrast, microvesicles, are produced directly through the outward budding and fission of membrane vesicles from the plasma membrane, and hence, their surface markers are largely dependent on the composition of the membrane of origin. Further, they tend to constitute a larger and more heterogeneous population of extracellular vesicles, ranging from 150 to 1000 nm in diameter. However, both types of vesicles have been shown to deliver functional mRNA, miRNA and proteins to recipient cells.

In some embodiments, the polynucleotides are delivered to the cells intracellularly via a gene gun, a microparticle or nanoparticle suitable for such delivery, transfection by electroporation, three-dimensional nanochannel electroporation, a tissue nanotransfection device, a liposome suitable for such delivery, or a deep-topical tissue nanoelectroinjection device. In some embodiments, a viral vector can be used. However, in other embodiments, the polynucleotides are not delivered virally.

Electroporation is a technique in which an electrical field is applied to cells in order to increase permeability of the cell membrane, allowing cargo (e.g., reprogramming factors) to be introduced into cells. Electroporation is a common technique for introducing foreign DNA into cells.

Tissue nanotransfection allows for direct cytosolic delivery of cargo (e.g., reprogramming factors) into cells by applying a highly intense and focused electric field through arrayed nanochannels, which benignly nanoporates the juxtaposing tissue cell members, and electrophoretically drives cargo into the cells.

In order to express a polypeptide or functional nucleic acid, the nucleotide coding sequence may be inserted into appropriate expression vector. Therefore, also disclosed is a non-viral vector comprising a polynucleotide comprising nucleic acid sequences disclosed herein, wherein the nucleic acid sequences are operably linked to an expression control sequence. In some embodiments, the nucleic acid sequences are operably linked to a single expression control sequence. In other embodiments, the nucleic acid sequences are operably linked to two or more separate expression control sequences.

Methods to construct expression vectors containing genetic sequences and appropriate transcriptional and translational control elements are well known in the art. These methods include in vitro recombinant DNA techniques, synthetic techniques, and in vivo genetic recombination. Such techniques are described in Sambrook et al., Molecular Cloning, A Laboratory Manual (Cold Spring Harbor Press, Plainview, N. Y., 1989), and Ausubel et al., Current Protocols in Molecular Biology (John Wiley & Sons, New York, N.Y., 1989).

Expression vectors generally contain regulatory sequences necessary elements for the translation and/or transcription of the inserted coding sequence. For example, the coding sequence is preferably operably linked to a promoter and/or enhancer to help control the expression of the desired gene product.

Promoters used in biotechnology are of different types according to the intended type of control of gene expression. They can be generally divided into constitutive promoters, tissue-specific or development-stage-specific promoters, inducible promoters, and synthetic promoters.

Constitutive promoters direct expression in virtually all tissues and are largely, if not entirely, independent of environmental and developmental factors. As their expression is normally not conditioned by endogenous factors, constitutive promoters are usually active across species and even across kingdoms. Examples of constitutive promoters include CMV, EF1a, SV40, PGK1, Ubc, Human beta actin, and CAG.

Tissue-specific or development-stage-specific promoters direct the expression of a gene in specific tissue(s) or at certain stages of development. For plants, promoter elements that are expressed or affect the expression of genes in the vascular system, photosynthetic tissues, tubers, roots and other vegetative organs, or seeds and other reproductive organs can be found in heterologous systems (e.g. distantly related species or even other kingdoms) but the most specificity is generally achieved with homologous promoters (i.e. from the same species, genus or family). This is probably because the coordinate expression of transcription factors is necessary for regulation of the promoter's activity.

The performance of inducible promoters is not conditioned to endogenous factors but to environmental conditions and external stimuli that can be artificially controlled. Within this group, there are promoters modulated by abiotic factors such as light, oxygen levels, heat, cold and wounding. Since some of these factors are difficult to control outside an experimental setting, promoters that respond to chemical compounds, not found naturally in the organism of interest, are of particular interest. Along those lines, promoters that respond to antibiotics, copper, alcohol, steroids, and herbicides, among other compounds, have been adapted and refined to allow the induction of gene activity at will and independently of other biotic or abiotic factors.

The two most commonly used inducible expression systems for research of eukaryote cell biology are named Tet-Off and Tet-On. The Tet-Off system makes use of the tetracycline transactivator (tTA) protein, which is created by fusing one protein, TetR (tetracycline repressor), found in Escherichia coli bacteria, with the activation domain of another protein, VP16, found in the Herpes Simplex Virus. The resulting tTA protein is able to bind to DNA at specific TetO operator sequences. In most Tet-Off systems, several repeats of such TetO sequences are placed upstream of a minimal promoter such as the CMV promoter. The entirety of several TetO sequences with a minimal promoter is called a tetracycline response element (TRE), because it responds to binding of the tetracycline transactivator protein tTA by increased expression of the gene or genes downstream of its promoter. In a Tet-Off system, expression of TRE-controlled genes can be repressed by tetracycline and its derivatives. They bind tTA and render it incapable of binding to TRE sequences, thereby preventing transactivation of TRE-controlled genes. A Tet-On system works similarly, but in the opposite fashion. While in a Tet-Off system, tTA is capable of binding the operator only if not bound to tetracycline or one of its derivatives, such as doxycycline, in a Tet-On system, the rtTA protein is capable of binding the operator only if bound by a tetracycline. Thus the introduction of doxycycline to the system initiates the transcription of the genetic product. The Tet-On system is sometimes preferred over Tet-Off for its faster responsiveness.

In some embodiments, the nucleic acid sequences disclosed herein are operably linked to the same expression control sequence. Alternatively, internal ribosome entry sites (IRES) elements can be used to create multigene, or polycistronic, messages. IRES elements are able to bypass the ribosome scanning model of 5′ methylated Cap dependent translation and begin translation at internal sites. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Disclosed are non-viral vectors containing one or more polynucleotides disclosed herein operably linked to an expression control sequence. Examples of such non-viral vectors include the oligonucleotide alone or in combination with a suitable protein, polysaccharide or lipid formulation. Non-viral methods present certain advantages over viral methods, with simple large scale production and low host immunogenicity being just two. Previously, low levels of transfection and expression of the gene held non-viral methods at a disadvantage; however, recent advances in vector technology have yielded molecules and techniques with transfection efficiencies similar to those of viruses.

Examples of suitable non-viral vectors include, but are not limited to pIRES-hrGFP-2a, pCMV6, pMAX, pCAG, pAd-IRES-GFP, and pCDNA3.0.

The compositions disclosed can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

### Therapeutic EVs

Also disclosed are EVs produced ex vivo and loaded with therapeutic cargo for use in treating NF1. The disclosed EVs can in some embodiments be any vesicle that can be secreted by a cell. Cells secrete extracellular vesicles (EVs) with a broad range of diameters and functions, including apoptotic bodies (1-5 μm), microvesicles (100-1000 nm in size), and vesicles of endosomal origin, known as exosomes (50-150 nm).

In some embodiments, the donor cells can be any donor cell able to produce EVs, including (but not limited to) skin cells (e.g., fibroblasts, keratinocytes, skin stem cells), adipocytes, dendritic cells, peripheral blood mononuclear cells (PBMC), pancreatic cells (e.g., ductal epithelial cells), liver cells (e.g., hepatocytes), immune cells (e.g., T cells, macrophages, myeloid derived suppressor cells).

The disclosed extracellular vesicles may be prepared by methods known in the art. For example, the disclosed extracellular vesicles may be prepared by expressing in a eukaryotic cell an mRNA that encodes the cell-targeting ligand. In some embodiments, the cell also expresses an mRNA that encodes a therapeutic cargo. The mRNA for the cell-targeting ligand and the therapeutic cargo may be expressed from vectors that are transfected into suitable production cells for producing the disclosed EVs. The mRNA for the cell-targeting ligand and the therapeutic cargo may be expressed from the same vector (e.g., where the vector expresses the mRNA for the cell-targeting ligand and the therapeutic cargo from separate promoters), or the mRNA for the cell-targeting ligand and the therapeutic cargo may be expressed from separate vectors. The vector or vectors for expressing the mRNA for the cell-targeting ligand and the therapeutic cargo may be packaged in a kit designed for preparing the disclosed extracellular vesicles.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable . . .

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The herein disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

### Therapeutic Cargo

The disclosed extracellular vesicles may be loaded with a therapeutic agent, where the extracellular vesicles deliver the agent to SCs. Suitable therapeutic agents include but are not limited to therapeutic drugs (e.g., small molecule drugs), therapeutic proteins, and therapeutic nucleic acids (e.g., therapeutic RNA or DNA). In some embodiments, the disclosed extracellular vesicles comprise a therapeutic RNA or DNA (also referred to herein as a “cargo RNA” or “cargo DNA”). In particular embodiments, the cargo is neurofibromin 1 of a NF1 gene.

For example, in some embodiments a cell-targeting protein also includes an RNA-domain (e.g., at a cytosolic C-terminus of the fusion protein) that binds to one or more RNA-motifs present in the cargo RNA in order to package the cargo RNA into the extracellular vesicle, prior to the extracellular vesicles being secreted from a cell. Likewise, in some embodiments a cell-targeting protein also includes a DNA-domain (e.g., at a cytosolic C-terminus of the fusion protein) that binds to one or more DNA-motifs present in the cargo DNA in order to package the cargo DNA into the extracellular vesicle, prior to the extracellular vesicles being secreted from a cell. As such, the protein may function as both of a “cell-targeting protein” and a “packaging protein.” In some embodiments, the packaging protein may be referred to as extracellular vesicle-loading protein or “EV-loading protein.”

The cargo RNA or cargo DNA of the disclosed extracellular vesicles may be of any suitable length. For example, in some embodiments the cargo RNA or cargo DNA may have a nucleotide length of at least about 10 nt, 20 nt, 30 nt, 40 nt, 50 nt, 100 nt, 200 nt, 500 nt, 1000 nt, 2000 nt, 5000 nt, or longer. In other embodiments, the cargo RNA may have a nucleotide length of no more than about 5000 nt, 2000 nt, 1000 nt, 500 nt, 200 nt, 100 nt, 50 nt, 40 nt, 30 nt, 20 nt, or 10 nt. In even further embodiments, the cargo RNA may have a nucleotide length within a range of these contemplated nucleotide lengths, for example, a nucleotide length between a range of about 10 nt-5000 nt, or other ranges. The cargo RNA or cargo DNA of the disclosed extracellular vesicles may be relatively long.

In some embodiments, the therapeutic cargo is a membrane-permeable pharmacological compound that is loaded into the EV after it is secreted by the cell.

To achieve loading of small RNAs into EVs, transfection-based approaches have been proposed. Other reports have shown that using vector-induced expression of small RNAs in cells, small RNA loading into EVs can be achieved. Alternatively, EV donor cells may be transfected with small RNAs directly. Incubation of tumor cells with chemotherapeutic drugs is also another method to package drugs into EVs. To stimulate formation of drug-loaded EVs, cells are irradiated with ultraviolet light to induce apoptosis. Alternative approaches such as fusogenic liposomes also leads loading drugs into EVs.

In some embodiments, the therapeutic cargo is loaded into the EVs by diffusion via a concentration gradient.

### Methods

Disclosed herein are methods for delivering diagnostic or therapeutic cargo to Schwann cells using the disclosed EVs. Therefore, also disclosed herein is a method for treating any disease or condition associated with Schwann cells.

For example, Schwannoma is a rare type of tumor that forms in the nervous system. Schwannoma tumors are often benign, which means they are not cancer. But, in rare cases, they can become cancer.

Neurofibromatosis type I (NF1) is an autosomal dominant genetic condition caused by mutations in the neurofibromin 1 (NF1) gene in Schwann cells (SCs). NF1, also called von Recklinghausen's disease is characterized by the development of multiple noncancerous (benign) tumors of nerves and skin (neurofibromas) and areas of abnormal skin color (pigmentation). Areas of abnormal skin pigmentation typically include pale tan or light brown discolorations (cafe-au-lait spots), freckling in atypical locations such as under the arms (axillary region) or in the groin (inguinal region). Such abnormalities of skin pigmentation are often evident by one year of age and tend to increase in size and number over time.

At birth or early childhood, affected individuals may have relatively large, benign tumors that consist of bundles of nerves and other tissue (plexiform neurofibromas). Individuals with NF1 may also develop benign nodules on the colored regions of the eyes (Lisch nodules), or tumors in the nerves of the visual pathway (optic pathway gliomas). More rarely, affected individuals may develop certain malignant (cancerous) tumors.

NF1 may also be characterized by an unusually large head size (macrocephaly) and relatively short stature. Additional abnormalities may also be present, such as episodes of uncontrolled electrical activity in the brain (seizures); learning disabilities, and attention deficits; speech difficulties; abnormally increased activity (hyperactivity); and skeletal malformations, including progressive curvature of the spine (scoliosis), bowing of the lower legs (pseudoarthrosis), and improper development of certain bones. Associated symptoms and findings may vary greatly in range and severity from person to person, even within the same family. Most people with NF1 have normal intelligence but learning disabilities appear in about 50% of children with NF1.

According to the National Institutes of Health (NIH) Consensus Conference in 1987, a clinical diagnosis of NF1 may be made if patients demonstrate at least two of the following: (1) Six or more café-au-lait spots of at least 5 millimeters [mm] in size (before puberty) or 15 mm in size (after puberty); (2) Freckling in the underarms (axillary) or groin (inguinal) regions; (3) Abnormal clumps of pigment on the colored portion of the eye (Lisch nodules); (4) Certain abnormalities of bone development in the head (sphenoid wing dysplasia) or abnormal bowing of bones (pseudoarthrosis); (5) Two or more neurofibromas of any type or one plexiform neurofibroma; (6) An affected parent, sibling, or child with confirmed NF1.

Symptoms of NF1 usually begin during childhood, and a definite diagnosis can often be made by four years of age or younger, depending on the circumstance. The disorder is progressive across the lifetime. In some cases, NF1 symptoms have been described to worsen during puberty, pregnancy, or when hormonal changes occur, though this correlation remains incompletely understood. The range and severity of NF1 symptoms varies greatly among affected individuals, and the rate of progression of this disorder is not predictable. However, a majority of patients (approximately 60%) are described as having a “mild” form of the condition.

Often the first sign of NF1 is the appearance of multiple brown spots on the skin (café-au-lait macules) or freckling in the underarm (axillary) or groin (inguinal) regions, which may occur as early as birth or infancy. Lisch nodules may also be present early in life, and are highly suggestive of an NF1 diagnosis, as they occur in approximately 97% of affected individuals.

Multiple noncancerous (benign) tumors (neurofibromas) develop in NF1 along the linings of the nerves (sheath) under the skin or in deeper areas of the body. Neurofibromas may form in any organ in the body. Skin (cutaneous) neurofibromas, or less discrete neurofibromas (plexiform neurofibromas) may cause disfigurement. Occasionally, tumors may develop in the brain, on the nerves exiting the brain, and/or on the spinal cord. The total number of neurofibromas in an adult may range from a few to hundreds or even thousands, and the number of these tumors tends to increase with age. Pain may occur from an affected peripheral nerve, or as a result of regional mass effect on adjacent structures. In 8-15% of affected individuals, neurofibromas may transform to become cancerous (malignant peripheral nerve sheath tumors), which are associated with pain, weight loss, night sweats, and require urgent evaluation and treatment.

Approximately 15% of people with NF1 develop brain tumors (gliomas), which nearly always develop during childhood. These frequently develop on the nerves of the eye (optic gliomas), and may affect vision or potentially lead to blindness. Additionally, a variety of other tumors may develop in patients with NF1, including gastrointestinal stromal tumors (GIST). In women with NF1, there is a 3.5-fold increased risk of developing breast cancer and a five-fold increased risk of developing breast cancer before the age of 50 years of age.

Orthopedic problems may develop with NF1, including curvature of the spine (scoliosis), abnormal cranial bone growth (sphenoid wing dysplasia), or a condition characterized by loss of bone tissue, fractures, and abnormal healing and bowing of weight-bearing long bones (pseudoarthrosis). Additionally, disorders of bone density (osteopenia and osteoporosis) are more common in people with NF1 than in the general population. The process by which these conditions develop is not fully understood, but has been associated with decreased activated vitamin D levels, increased parathyroid hormone levels, and increased markers of bone breakdown. People with NF1 tend to be below average in height, below average in muscle strength, and above average in head size for age.

High blood pressure (hypertension) is seen with greater frequency in the NF1 population than the general population. While the cause for this is not certain, some cases may not directly relate to NF1, but rather to associated changes in the blood vessels leading to the kidneys (renal artery stenosis). More rarely, patients with NF1 are at risk of developing tumors of the adrenal gland (pheochromocytoma), which may cause severely elevated blood pressure without treatment.

Sexual development may be delayed or may occur early (precocious puberty) in individuals with NF1. (For more information on this disorder, choose “precocious puberty” as your search term in the Rare Disease Database.) In addition, over 50% of individuals with NF1 experience learning disabilities, such as attention deficit hyperactivity disorder (ADHD). Seizures may also occur. Other symptoms include headache, numbness, and/or weakness.

In the localized form of NF1, known as segmental neurofibromatosis, abnormal pigmentation and/or tumors may be limited to one area of the body.

Neurofibromatosis 2 (NF2) is a rare disorder that is genetically distinct from NF1. NF2 is characterized by benign tumors on both auditory nerves (vestibular schwannomas) and in other areas of the body. Other tumors of the central nervous system may develop including meningiomas and/or ependymomas. Individuals with NF2 do not typically have cafe-au-lait macules or abnormal skin freckling. Other symptoms of NF2 may include problems with balance, buzzing or ringing in the ears (tinnitus), progressive hearing loss, or facial weakness. In some embodiments, the disclosed compositions and methods can be used to treat NF2.

The disclosed EVs may be administered to a subject by any suitable means. Administration to a human or animal subject may be selected from parenteral, intramuscular, intracerebral, intravascular, subcutaneous, or transdermal administration. Typically the method of delivery is by injection. Preferably the injection is intramuscular or intravascular (e.g. intravenous). A physician will be able to determine the required route of administration for each particular patient.

The EVs are preferably delivered as a composition. The composition may be formulated for parenteral, intramuscular, intracerebral, intravascular (including intravenous), subcutaneous, or transdermal administration. Compositions for parenteral administration may include sterile aqueous solutions which may also contain buffers, diluents and other suitable additives. The EVs may be formulated in a pharmaceutical composition, which may include pharmaceutically acceptable carriers, thickeners, diluents, buffers, preservatives, and other pharmaceutically acceptable carriers or excipients and the like in addition to the EVs.

Parenteral administration is generally characterized by injection, such as subcutaneously, intramuscularly, or intravenously. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof. Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include sodium chloride injection, ringers injection, isotonic dextrose injection, sterile water injection, dextrose and lactated ringers injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone.

Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment. The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations can be packaged in an ampoule, a vial or a syringe with a needle. All preparations for parenteral administration should be sterile, as is known and practiced in the art.

A therapeutically effective amount of composition is administered. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual constructs, and can generally be estimated based on EC50s found to be effective in vitro and in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific construct, the age, weight and condition of the subject to be treated, the severity of the disease and the frequency and route of administration. Different dosages of the construct may be administered depending on whether administration is by intramuscular injection or systemic (intravenous or subcutaneous) injection.

Preferably, the dose of a single intramuscular injection is in the range of about 5 to 20 μg. Preferably, the dose of single or multiple systemic injections is in the range of 10 to 100 mg/kg of body weight.

Due to construct clearance (and breakdown of any targeted molecule), the patient may have to be treated repeatedly, for example once or more daily, weekly, monthly or yearly. Persons of ordinary skill in the art can easily estimate repetition rates for dosing based on measured residence times and concentrations of the construct in bodily fluids or tissues. Following successful treatment, it may be desirable to have the patient undergo maintenance therapy, wherein the construct is administered in maintenance doses, ranging from 0.01 mg/kg to 100 mg per kg of body weight, once or more daily, to once every 20 years.

### Example Embodiments

Embodiment 1. A composition comprising extracellular vesicles (EVs) produced from donor cells engineered to express NRG1, NRG2, or a combination thereof.

Embodiment 2. The composition of embodiment 1, wherein the donor cells are autologous.

Embodiment 3. The composition of embodiment 1 or 2, wherein the donor cells are skin cells.

Embodiment 4. The composition of any one of embodiments 1 to 3, wherein the EVs encapsulate a therapeutic cargo.

Embodiment 5. The composition of embodiment 4, wherein the therapeutic cargo comprises NF1 or a nucleic acid encoding neurofibromin.

Embodiment 6. The composition of embodiment 1, wherein the EVs selectively target Schwann cells.

Embodiment 7. A method for selectively delivering a therapeutic cargo to Schwann cells in a subject, comprising administering to the subject an effective amount of embodiment 4.

Embodiment 8. A method of treating neurofibromatosis type 1 (NF1) in a subject, comprising administering to the subject an effective amount of a composition of any one of embodiments 1 to 6.

Embodiment 9. A method for treating neurofibromatosis type 1 (NF1) in a subject, comprising delivering intracellularly into skin cells of the subject a polynucleotide comprising nucleic acid sequences encoding NRG1, NRG2, or a combination thereof, and a nucleic acid sequence encoding neurofibromin, wherein the skin cells produce EVs decorated with NRG1, NRG2, or a combination thereof and encapsulating neurofibromin as a therapeutic cargo.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Develop Designer EVs for Targeted Delivery

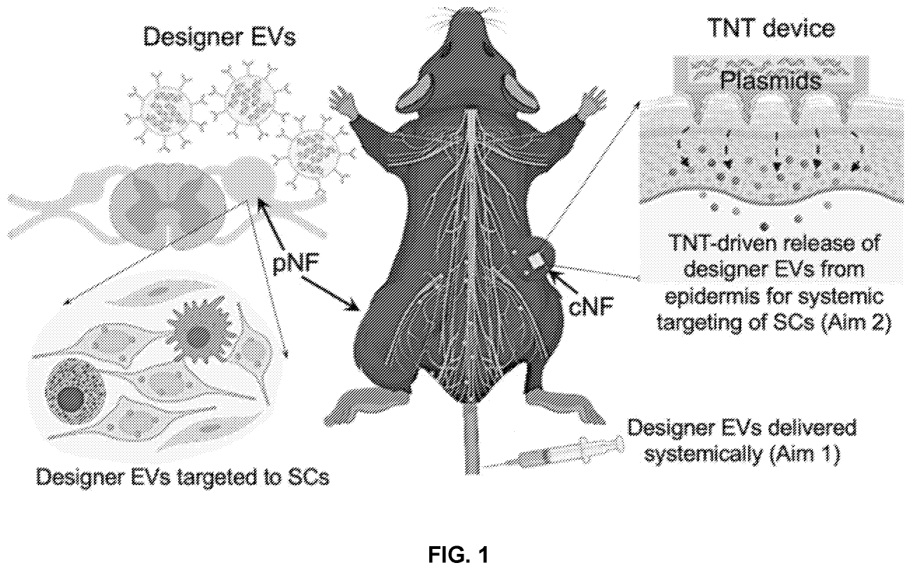

Nanotransfection is used to engineer fibroblasts to produce EVs with tropism for SCs, and deliver therapeutic payloads to SCs systemically (FIG. 1). Tunneling nanotubes (TNT) is also used to engineer skin patches to release (in-situ) designer EVs with tropism to SCs, and deliver therapeutic payloads to SCs systemically (FIG. 1).

NF1 is driven by mutations/deletions of the NF1 gene in SCs. As NF1 has many key functions (e.g., modulation of Ras-GTPase), such mutations/deletions lead to neurofibromas. As such, gene therapies have been studied to recover NF1 function. Retroviral and adeno-associated viral vectors have been used to transfect the GAP-related domain of NF1 to recover function. However, these studies often yield low efficiencies. Moreover, viral vectors fail to carry full-length NF1 due to capsid size restrictions.

Viral vectors have become the gold standard for gene therapies. However, while promising, viruses have several limitations beyond capsid size constraints. Viral vector-induced immunity, for example, could prevent redosing or pose biosafety concerns. EVs have thus emerged as promising therapeutic carriers for gene therapies. Compared to most carrier systems, viral or synthetic, EVs are able to package large cargos, show improved biocompatibility, reduced immunogenicity, enhanced stability, and an innate ability to pass through biological barriers. As such, a substantial amount of research is being devoted to engineering therapeutic EVs for different diseases. However, currently there is a paucity of research on designer EVs for the delivery of therapeutic payloads for NF1.

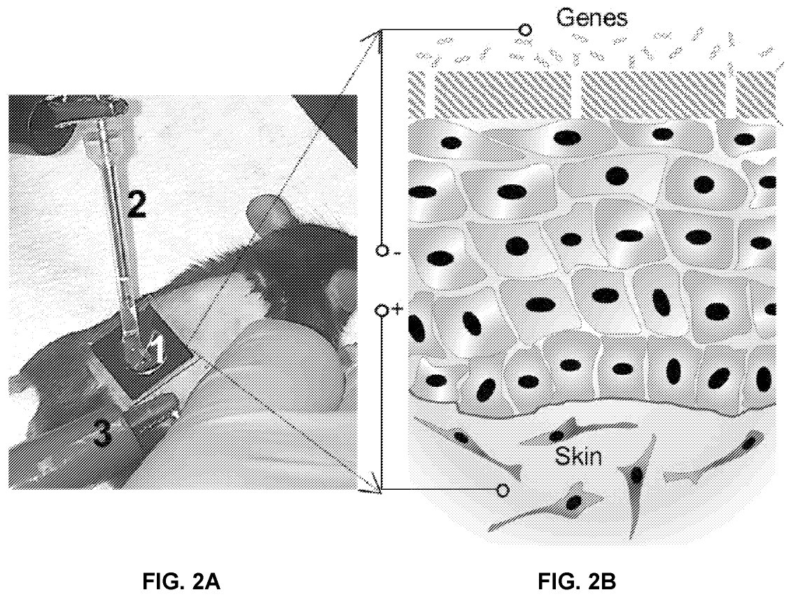

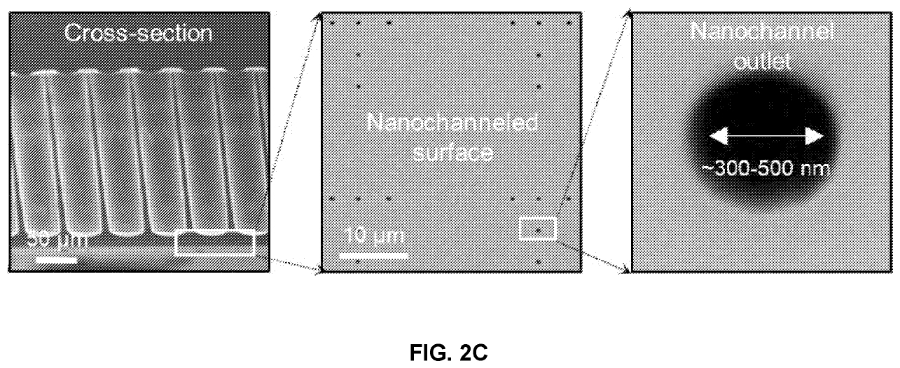

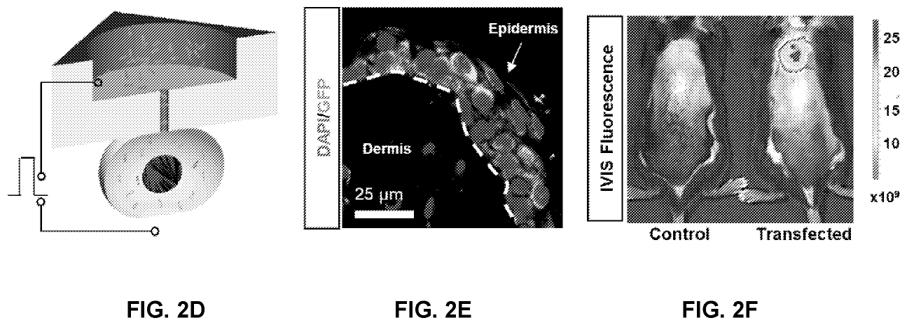

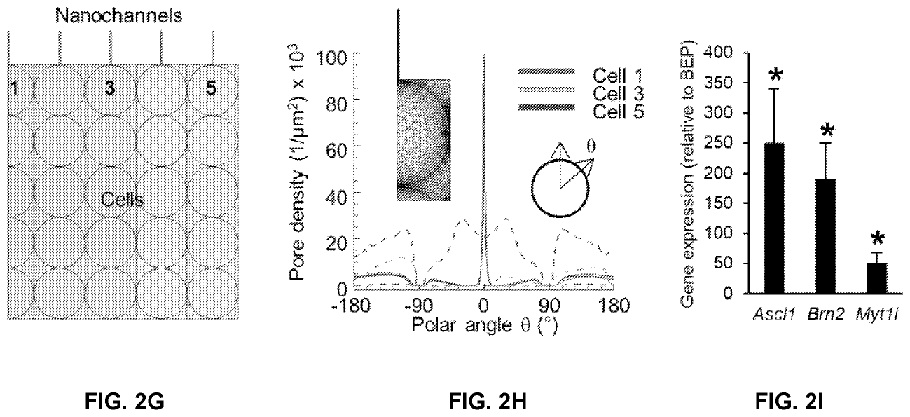

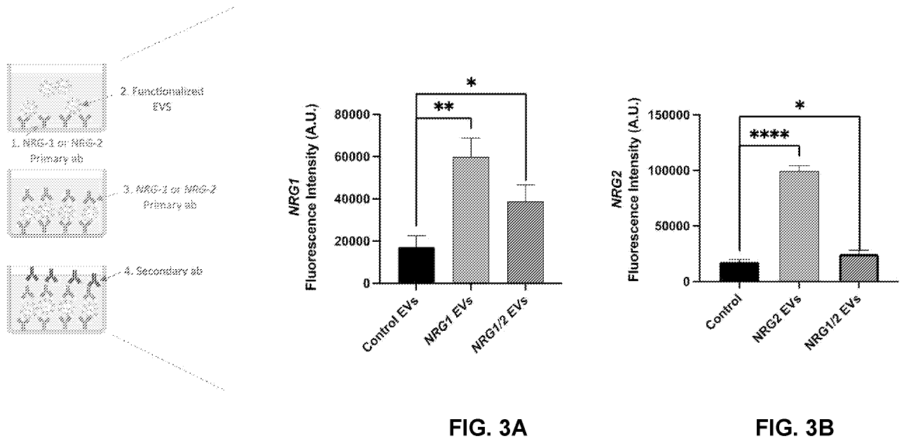

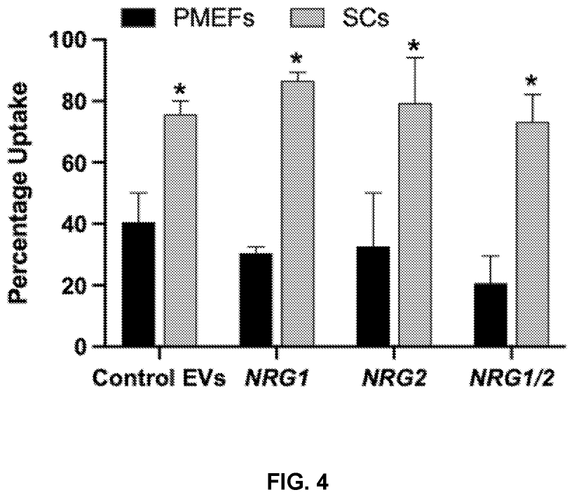

TNT can be used for non-viral (i.e., no capsid size restriction) gene delivery in vivo. TNT uses Silicon nanochannels and electric fields to deliver cargo into solid tissues tissue in a fast (˜100 milliseconds), efficient, and benign manner (FIG. 2). This is accomplished via a combination of nanoscale electroporation and electrophoresis. In silico and in vivo studies validate the superiority of TNT vs. standard bulk electroporation (BEP). TNT will be used to drive gene therapies for cutaneous (cNFs) and plexiform neurofibromas (pNFs) in mice.

In addition to enabling direct gene delivery to solid tissue, TNT can also drive the release of designer EVs from the epidermis, loaded with copies and transcripts of the TNT′d gene. Studies suggest that TNT-treated skin can produce EVs that could potentially be used to amplify the transfection beyond the skin. In addition to systemic delivery of designer EVs made ex vivo, here TNT is used to “force” the epidermis to produce SC-targeting designer EVs (in vivo) that can be directed to cNFs and pNFs, systemically, via specific receptor-ligand interactions.

EV-based approaches to deploy gene therapies for NF1 are in some embodiments produced by dispatching SC-targeting EVs from the skin. For example, in addition to directly delivering therapeutic genes to cNFs, TNT is used to program the epidermis to systemically release designer EVs with SC tropism.

Full-length NF1 is used as model cargo, but the proposed EV technology could be used to deliver different types of therapeutic cargo (e.g., CRISPR/Cas9).

### Example 2: Develop Designer EVs for SC Targeted Delivery

Designer EV formulations are developed with enhanced tropism for SCs. This is done by nanotransfecting mouse dermal fibroblasts (MDFs) with plasmids to overexpress SC-targeting ligands (HRG1/2, NRG1), or drive conversions into SCs (SOX10, EGR2). The working hypothesis is that nanotransfecting MDFs with SC-targeting ligand plasmids will lead to release of functionalized EVs that will preferentially bind to SCs via ligand-receptor interactions (HRG1/2-ErbB2/3, NRG1-EGF/ErbB3). Thus, methods are needed to impart SC tropism to EVs derived from more readily abundant cells.

EVs Decorated with Targeting Ligands:

Nanotransfection platforms are fabricated and used to deliver plasmids for SC-targeting ligands to MDFs as described before. Briefly, MDFs (ScienCell) are plated in direct contact with the nanochannels, and a pulsed electric field (˜250V, 10 ms pulses, 10 pulses) are used to deliver the plasmids. Sham plasmids with the same backbone are used as controls. Plasmids are transfected individually or equimollary mixed in dual permutations. EVs are isolated from the supernatant at 6-72 h using an ExoQuick kit. EV functionalization with targeting ligands are evaluated with western blot (WB). NanoSight is used to quantify EV concentration and size.

### Selective Uptake:

Selective SC uptake of designer EV formulations is assessed in co-cultures of SCs and MDFs. SCs (ATCC) and MDFs are mixed at a 1:1 ratio. The cells and EVs are labeled with fluorophores of different wavelengths (˜490, 560, and 650 nm). Co-cultures are exposed to ˜109-1010 EVs/ml, and selective uptake by SCs vs. MDFs are evaluated via confocal microscopy. EVs derived from SCs are used as positive control.

### Biodistribution:

To identify EV formulations with enhanced tropism for SCs in cNFs/pNFs, a murine model of NF1 is used, in which the Nf1 alleles are inactivated in SOX10+ cells, leading to the formation of cNFs and pNFs. Briefly, homozygous Nf1^{fl/fl} mice (Stock No: 017640, JAX) are mated with tamoxifen-inducible SOX10-CreERT2 mice (Stock No: 027651, JAX). From the offspring, Nf1^{fl/-}:SOX10-CreERT2^{+/0} mice are mated with Nf1^{fl/fl} mice. Approximately 25% of the offspring have a homozygous genotype for the Nf1flox allele and hemizygous for the SOX10-CreERT2 allele (Nf1^{fl/fl}:SOX10-CreERT2^{+/0}) and are used as the experimental line. Offspring homozygous for the Nf1flox allele and null for SOX10-CreERT2 allele (Nf1^{fl/fl}:SOX10-CreERT2^{0/0}) are used as controls. Mice are treated with tamoxifen at ˜1 month of age. EV formulations are injected via tail vein ˜6 months after tamoxifen induction, once cNF/pNF lesions/symptoms (scruffy fur, hunched posture, limping, limb paralysis) are confirmed. EVs derived from SCs are used as positive control. The EVs are fluorescently tagged with MemGlow. The mice are injected with a bolus of ˜10¹² EVs/gram of weight daily, and mice injected 1-5 times are compared. The mice are euthanized 24 hours after the last injection, and cNF lesions, spinal cord/sciatic nerves (to inspect pNFs), liver, lungs, spleen, and kidneys are collected and imaged by IVIS to evaluate EV distribution. The tissues are subsequently processed for histology. Neurofibromas are immunostained for S100β, GAP43, SOX10, Iba1, and mast cells. The presence of EVs in tissue sections is quantified by confocal imaging.

Delivering NF1 to cNFs/pNFs:

Once an optimum set of ligands is identified, EVs loaded with full-length NF1 as model cargo are produced. MDFs are co-nanotransfected (1:1 ratio) with NF1 (˜13.4 kb, Origene) and optimized ligand plasmids. Positive control EVs are prepared by delivering NF1 into SCs. Negative control EVs are prepared by co-delivering sham+ligand plasmids. Designer EVs are collected from the supernatant at 6-72 h, and EV functionalization and NF1 loading are evaluated by WB and qRT-PCR. Selective uptake by SCs of NF1-loaded EVs is evaluated. qRT-PCR is used to evaluate NF1 expression in SCs. EVs are delivered to tamoxifen-treated Nf1fl/fl: SOX10-CreERT2^{+/0} mice via tail vein with a bolus of ˜10¹² EVs/gram of weight daily, and mice injected 1-5 times are compared. Biodistribution is assessed. NF1 delivery cNFs/pNFs, and function, are evaluated by qRT-PCR for NF1, and immunostaining for neurofibromin, p-ERK, and quantification of SOX10+ and mast cells. Additional assessments include quantification of the number and volume of neurofibromas, as well as TUNEL and BrdU staining. To verify if EVs carried NF1, laser capture microdissection (LCM) is used to isolate fluorescently tagged portions of tissue sections (indicative of tagged EV accumulation), and PCR/qRT-PCR are used to quantify NF1 plasmid/mRNA at that location.

### Example 3: Develop TNT Protocols for In Situ Deployment of SC-Targeting EVs

An alternative approach is tested to deploy EV-based therapies for NF1 by using TNT to force the epidermis to produce SC-targeting EVs to treat neurofibromas. Using the skin as an in-situ source of therapeutic EVs obviates the need for isolation/purification, thus potentially facilitating scale up and translation. The working hypotheses are that TNT-based co-delivery of NF1+optimized formulations of SC-targeting ligands, can (1) result in direct delivery of NF1 to SCs in cNFs, and (2) release of epidermal EVs loaded with NF1, and “decorated” with SC-targeting ligands. Continuous drainage of such EVs into peripheral lymph nodes or systemic circulation is thus likely to result in systemic spread beyond the skin and homing to SCs in cNFs and pNFs.

### TNT Device Fabrication:

TNT platforms are fabricated using wafer-scale cleanroom procedures, from 4″ Si wafers, as shown before. Briefly, nanochannels (˜300 nm Ø, ˜20 μm deep) are etched into the Si using a combination of projection and contact lithography with deep reactive ion etching. Characterization conducted at each step of the process via electron microscopy guarantees the device quality. Processed wafers are diced into 1 cm² devices, which are affixed to a plastic casing to form the plasmid reservoir.

### Transfections:

Plasmids for NF1 and optimized formulations for targeting ligands are co-TNT (1:1 ratio). TNT is conducted directly on cNFs or normal skin of tamoxifen-induced Nf1^{fl/fl}. SOX10-CreERT2^{+/0} mice. The skin is naired prior to TNT. TNT with sham plasmids alone, and sham+targeting ligand plasmids serve as control. Plasmids are delivered by applying a pulsed electric field (250V, 10 ms pulses, 10 pulses) across a pair of electrodes located between the plasmid reservoir and the skin. Since the duration of the TNT procedure is only ˜100 ms, 1-4 spots are TNT per mouse. To evaluate redosing effects, TNT is conducted weekly for 1-5 weeks. The mice are euthanized and TNT′d skin, cNF lesions, spinal cord/nerves, liver, spleen, kidneys, and lungs are collected. To trace epidermal EVs, the skin is pre-TNT with EV tracker plasmids (pCT-CD63-GFP, Systems Bio) 24 hours prior to TNT of NF1 and targeting ligand plasmids.

### TNT Outcomes:

Successful delivery/expression of NF1 to cNFs or normal skin is evaluated by LCM of epidermis and dermis, followed by PCR/qRT-PCR and immunostaining to evaluate plasmid delivery and expression (mRNA and protein level). EVs are isolated from skin biopsies, and decoration and loading is characterized by WB and qRT-PCR. The biodistribution of CD63-GFP-tagged EVs is evaluated. EV-based delivery/expression of NF1 in cNFs that were not TNT'd directly, and in pNFs, are characterized by LCM/qRT-PCR to analyze GFP+ regions where epidermal EVs accumulated. Functional assessments are also performed in cNFs and pNFs.

### Example 4: Protocol for Creating the EVs

EVs were isolated from culture media 24 hours after transfection of the donor cells. Culture media was centrifuged at 2,000 g for 30 min at 4° C. to remove death cells and debris. After centrifugation, cell-free culture media was filtered and concentrated using the Vivaspin filters (Sartorius, 76408-886) with a 300 kDa molecular weight cutoff. EVs were subsequently isolated using one of two methods: 1) concentrated solution containing EVs was then subjected to size exclusion chromatography using a qEV Original SEC column onto a qEV Automatic Fraction Collector (Izon Science Ltd.), and 3 EV-rich fractions were collected and stored for subsequent analysis; or 2) total exosome isolation reagent (Thermo Fisher Scientific, 44-783-59) was added to the supernatant containing the cell-free culture media following manufacturer instructions. All EVs were characterized in solution by measuring their concentration and size distribution via nanoparticle tracking analysis (NTA) technique and quantitative qRT-PCR was used to verify the packing of the molecular cargo inside the EVs prior any experiment.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A composition comprising extracellular vesicles (EVs) produced from donor cells engineered to express NRG1, NRG2, or a combination thereof.

2. The composition of claim 1, wherein the donor cells are autologous.

3. The composition of claim 1, wherein the donor cells are skin cells.

4. The composition of claim 1, wherein the EVs encapsulate a therapeutic cargo.

5. The composition of claim 4, wherein the therapeutic cargo comprises NF1 or a nucleic acid encoding neurofibromin.

6. The composition of claim 1, wherein the EVs selectively target Schwann cells.

7. A method for selectively delivering a therapeutic cargo to Schwann cells in a subject, comprising administering to the subject an effective amount of claim 4.

8. A method of treating neurofibromatosis type 1 (NF1) in a subject, comprising administering to the subject an effective amount of a composition of claim 1.

9. A method for treating neurofibromatosis type 1 (NF1) in a subject, comprising delivering intracellularly into skin cells of the subject a polynucleotide comprising nucleic acid sequences encoding NRG1, NRG2, or a combination thereof, and a nucleic acid sequence encoding neurofibromin, wherein the skin cells produce EVs decorated with NRG1, NRG2, or a combination thereof and encapsulating neurofibromin as a therapeutic cargo.


SEQUENCE LISTING (8 sequences)

SEQ ID NO: 1 - DNA, 2106 bp, Homo sapiens
  source 1..2106: mol_type = other DNA
gagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccagccttgcctccccaattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgccaacctggattcactggagcaagatgtactgagaatgtgcccatgaaagtccaaaaccaagaaaaggcggaggagctgtaccagaagagagtgctgaccataaccggcatctgcatcgccctccttgtggtcggcatcatgtgtttggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtatgtgtcagccatgaccaccccggctcgtatgtcacctgtagatttccacacgccaagctcccccaaatcgcccccttcggaaatgtctccacccgtgtccagcatgacggtgtccatgccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttattttagcagttctgcaaatagaaaacaggaaaaa

SEQ ID NO: 2 - protein, 640 aa, Homo sapiens [sequence omitted]

SEQ ID NO: 3 - DNA, 2620 bp, Homo sapiens
  source 1..2620: mol_type = other DNA
gtacaaaaaagcagaagggccgtcaaggcccaccatgcggcaggtttgctgctcagcgctgccgccgccgccactggagaagggtcggtgcagcagctacagcgacagcagcagcagcagcagcgagaggagcagcagcagcagcagcagcagcagcgagagcggcagcagcagcaggagcagcagcaacaacagcagcatctctcgtcccgctgcgcccccagagccgcggccgcagcaacagccgcagccccgcagccccgcagcccggagagccgccgcccgttcgcgagccgcagccgccggcggcatgaggcgcgacccggcccccggcttctccatgctgctcttcggtgtgtcgctcgcctgctactcgcccagcctcaagtcagtgcaggaccaggcgtacaaggcacccgtggtggtggagggcaaggtacaggggctggtcccagccggcggctccagctccaacagcacccgagagccgcccgcctcgggtcgggtggcgttggtaaaggtgctggacaagtggccgctccggagcggggggctgcagcgcgagcaggtgatcagcgtgggctcctgtgtgccgctcgaaaggaaccagcgctacatctttttcctggagcccacggaacagcccttagtctttaagacggcctttgcccccctcgataccaacggcaaaaatctcaagaaagaggtgggcaagatcctgtgcactgactgcgccacccggcccaagttgaagaagatgaagagccagacgggacaggtgggtgagaagcaatcgctgaagtgtgaggcagcagccggtaatccccagccttcctaccgttggttcaaggatggcaaggagctcaaccgcagccgagacattcgcatcaaatatggcaacggcagaaagaactcacgactacagttcaacaaggtgaaggtggaggacgctggggagtatgtctgcgaggccgagaacatcctggggaaggacaccgtccggggccggctttacgtcaacagcgtgagcaccaccctgtcatcctggtcggggcacgcccggaagtgcaacgagacagccaagtcctattgcgtcaatggaggcgtctgctactacatcgagggcatcaaccagctctcctgcaagtgtcctgtgggatacaccggggacaggtgtcagcagttcgcaatggtcaacttctccaagcaccttggatttgaattaaaggaagccgaggagctgtaccagaagagggtcctgaccatcacgggcatctgcgtggctctgctggtcgtgggcatcgtctgtgtggtggcctactgcaagaccaaaaaacagcggaagcagatgcacaaccacctccggcagaacatgtgcccggcccatcagaaccggagcttggccaatgggcccagccacccccggctggacccagaggagatccagatggcagattatatttccaagaacgtgccagccacagaccatgtcatcaggagagaaactgagaccaccttctctgggagccactcctgttctccttctcaccactgctccacagccacacccacctccagccacagacacgagagccacacgtggagcctggaacgttctgagagcctgacttctgactcccagtcggggatcatgctatcatcagtgggtaccagcaaatgcaacagcccagcatgtgtggaggcccgggcaaggcgggcagcagcctacaacctggaggagcggcgcagggccaccgcgccaccctatcacgattccgtggactcccttcgcgactccccacacagcgagaggtacgtgtcggccctgaccacgcccgcgcgcctctcgcccgtggacttccactactcgctggccacgcaggtgccaactttcgagatcacgtcccccaactcggcgcacgccgtgtcgctgccgccggcggcgcccatcagttaccgcctggccgagcagcagccgttactgcggcacccggcgccccccggcccgggacccggacccgggcccgggcccgggcccggcgcagacatgcagcgcagctatgacagctactattaccccgcggcggggcccggaccgcggcgcgggacctgcgcgctcggcggcagcctgggcagcctgcctgccagccccttccgcatccccgaggacgacgagtacgagaccacgcaggagtgcgcgcccccgccgccgccgcggccgcgcgcgcgcggtgcgtcccgcaggacgtcggcggggccccggcgctggcgccgctcgcgcctcaacgggctggcggcgcagcgcgcacgggcggcgagggactcgctgtcgctgagcagcggctcgggcggcggctcagcctcggcgtcggacgacgacgcggacgacgcggacggggcgctggcggccgagagcacacctttcctgggcctgcgtggggcgcacgacgcgctgcgctcggactcgccgccactgtgcccggcggccgacagcaggacttactactcactggacagccacagcacgcgggccagcagcagacacagccgcgggccgcccccgcgggccaagcaggactcggcgccactctagggcctcatgggcccagctttcttgtac

SEQ ID NO: 4 - protein, 852 aa, Homo sapiens
MRQVCCSALPPPPLEKGRCSSYSDSSSSSSERSSSSSSSSSESGSSSRSSSNNSSISRPAAPPEPRPQQQPQPRSPAARRAAARSRAAAAGGMRRDPAPGFSMLLFGVSLACYSPSLKSVQDQAYKAPVVVEGKVQGLVPAGGSSSNSTREPPASGRVALVKVLDKWPLRSGGLQREQVISVGSCVPLERNQRYIFFLEPTEQPLVFKTAFAPLDTNGKNLKKEVGKILCTDCATRPKLKKMKSQTGQVGEKQSLKCEAAAGNPQPSYRWFKDGKELNRSRDIRIKYGNGRKNSRLQFNKVKVEDAGEYVCEAENILGKDTVRGRLYVNSVSTTLSSWSGHARKCNETAKSYCVNGGVCYYIEGINQLSCKCPVGYTGDRCQQFAMVNFSKHLGFELKEAEELYQKRVLTITGICVALLVVGIVCVVAYCKTKKQRKQMHNHLRQNMCPAHQNRSLANGPSHPRLDPEEIQMADYISKNVPATDHVIRRETETTFSGSHSCSPSHHCSTATPTSSHRHESHTWSLERSESLTSDSQSGIMLSSVGTSKCNSPACVEARARRAAAYNLEERRRATAPPYHDSVDSLRDSPHSERYVSALTTPARLSPVDFHYSLATQVPTFEITSPNSAHAVSLPPAAPISYRLAEQQPLLRHPAPPGPGPGPGPGPGPGADMQRSYDSYYYPAAGPGPRRGTCALGGSLGSLPASPFRIPEDDEYETTQECAPPPPPRPRARGASRRTSAGPRRWRRSRLNGLAAQRARAARDSLSLSSGSGGGSASASDDDADDADGALAAESTPFLGLRGAHDALRSDSPPLCPAADSRTYYSLDSHSTRASSRHSRGPPPRAKQDSAPL

SEQ ID NO: 5 - DNA, 1370 bp, Homo sapiens
  source 1..1370: mol_type = other DNA
agaccgactaggggactgggagcgggcggcgcggccatggcgggctgctgcgccgcgctggcggccttcctgttcgagtacgacacgccgcgcatcgtgctcatccgcagccgcaaagtggggctcatgaaccgcgccgtgcaactgctcatcctggcctacgtcatcggacctgcttttttaaaggctgcagaaaacttcactcttttggttaagaacaacatctggtatcccaaatttaatttcagcaagaggaatatccttcccaacatcaccactacttacctcaagtcgtgcatttatgatgctaaaacagatcccttctgccccatattccgtcttggcaaaatagtggagaacgcaggacacagtttccaggacatggccgtggagggaggcatcatgggcatccaggtcaactgggactgcaacctggacagagccgcctccctctgcttgcccaggtactccttccgccgcctcgatacacgggacgttgagcacaacgtatctcctggctacaatttcaggtttgccaagtactacagagacctggctggcaacgagcagcgcacgctcatcaaggcctatggcatccgcttcgacatcattgtgtttgggaaggcagggaaatttgacatcatccccactatgatcaacatcggctctggcctggcactgctaggcatggcgaccgtgctgtgtgacatcatagtcctctactgcatgaagaaaagactctactatcgggagaagaaatataaatatgtggaagattacgagcagggtcttgctagtgagctggaccagtgaggcctaccccacacctgggctctccacagccccatcaaagaacagagaggaggaggagggagaaatggccaccacatcaccccagagaaatttctggaatctgattgagtctccactccacaagcactcagggttccccagcagctcctgtgtgttgtgtgcaggatctgtttgcccactcggcccaggaggtcagcagtctgttcttggctgggtcaactctgcttttcccgcaacctggggttgtcgggggagcgctggcccgacgcagtggcactgctgtggctttcagggctggagctggctttgctcagaagcctcctgtctccagctctctccaggacaggcccagtcctctgaggcacggcggctctgttcaagcactttatgcggcaggggaggccgcctggctgcagtcactagacttgtagcaggcctgggctgcaggcttccccccgaccattccctgcagccatgcggcagagctggcatttctcctcagagaagcgctgtgctaaggtgatcgaggaccagacattaaagcgtgattttcttaa

SEQ ID NO: 6 - protein, 258 aa, Homo sapiens
MAGCCAALAAFLFEYDTPRIVLIRSRKVGLMNRAVQLLILAYVIGPAFLKAAENFTLLVKNNIWYPKFNFSKRNILPNITTTYLKSCIYDAKTDPFCPIFRLGKIVENAGHSFQDMAVEGGIMGIQVNWDCNLDRAASLCLPRYSFRRLDTRDVEHNVSPGYNFRFAKYYRDLAGNEQRTLIKAYGIRFDIIVFGKAGKFDIIPTMINIGSGLALLGMATVLCDIIVLYCMKKRLYYREKKYKYVEDYEQGLASELDQ

SEQ ID NO: 7 - DNA, 5615 bp, Homo sapiens
  source 1..5615: mol_type = other DNA
gcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggagtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtaggagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctctttaaaaaaaaaaaaaaaaaaaaaaaaaaaactttagaactgggtgcagtggctcatgcctgtaatcccagccagcactttgggaggctgagatgggaagatcacttgagcccagaattagagataagcctatggaaacatagcaagacactgtctctacaggggaaaaaaaaaaaagaaactgagccttaaagagatgaaataaattaagcagtagatccaggatgcaaaatcctcccaattcctgtgcatgtgctcttattgtaaggtgccaagaaaaactgatttaagttacagcccttgtttaaggggcactgtttcttgtttttgcactgaatcaagtctaaccccaacagccacatcctcctatacctagacatctcatctcaggaagtggtggtgggggtagtcagaaggaaaaataactggacatctttgtgtaaaccataatccacatgtgccgtaaatgatcttcactccttatccgagggcaaattcacaaggatccccaagatccacttttagaagccattctcatccagcagtgagaagcttccaggtaggacagaaaaaagatccagcttcagctgcacacctctgtccccttggatggggaactaagggaaaacgtctgttgtatcactgaagttttttgttttgtttttatacgtgtctgaataaaaatgccaaagttttttttca

SEQ ID NO: 8 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT